(19) United States
(12) Patent Application Publication
(10) Pub. No.: US 2023/0042882 A1
(43) Pub. Date: Feb. 9, 2023

(54) METHOD OF MAPPING AND MACHINE LEARNING FOR PATIENT-HEALTHCARE ENCOUNTERS TO PREDICT PATIENT HEALTH AND DETERMINE TREATMENT OPTIONS

(71) Applicant: Insight Direct USA, Inc., Tempe, AZ (US)

(72) Inventor: Michael Griffin, Wayland, MA (US)

(21) Appl. No.: 17/684,878

(22) Filed: Mar. 2, 2022

Related U.S. Application Data
(60) Provisional application No. 63/230,499, filed on Aug. 6, 2021.
(60) Provisional application No. 63/230,507, filed on Aug. 6, 2021.
(60) Provisional application No. 63/313,496, filed on Feb. 24, 2022.

(51) Int. Cl. G16H 50/30; G16H 10/60

(52) U.S. Cl. CPC G16H 50/30; G16H 10/60

(57) ABSTRACT

A health evaluator is configured to determine future patient health. The health evaluator receives pertinent health data regarding a patient and analyzes the pertinent health data by a health prediction machine learning model trained on baseline health data to generate predictive health data regarding an expected patient condition. The baseline health data is based at least in part on electronic medical records (EMRs). The health evaluator generates treatment information for the patient based on one or more sets of the predictive health data generated by the health evaluator.

### CROSS-REFERENCE TO RELATED APPLICATION(S)

This application claims priority to U.S. Provisional Application No. 63/230,499 filed Aug. 6, 2021, and entitled “EFFICIENT AND INTERPRETABLE GRAPH METHOD TO LEARN SEQUENCES OF PATIENT-HOSPITAL ENCOUNTERS THAT PREDICT PATIENT HEALTH,” and claims priority to U.S. Provisional Application No. 63/230,507 filed Aug. 6, 2021, and entitled “METHOD FOR DETERMINING PATIENT ACTIONS AND HEALTHCARE PROVIDER ACTIONS THAT SIMULTANEOUSLY MAXIMIZE PATIENT HEALTH AND MINIMIZE HEALTHCARE COSTS,” and claims priority to U.S. Provisional Application No. 63/313,496 filed Feb. 24, 2022 and entitled “METHOD OF MAPPING AND MACHINE LEARNING FOR PATIENT-HEALTHCARE ENCOUNTERS TO PREDICT PATIENT HEALTH,” the disclosures of which are hereby incorporated by reference in their entireties.

### BACKGROUND

This disclosure relates generally to predictive healthcare. More specifically, this disclosure relates to modeling healthcare encounters by directed graphs and training machine learning models based on the graphed encounters; this disclosure further relates to enhancing patient and provider actions using machine learning models.

Patient interactions with healthcare systems are commonly known as encounters. Encounters may be office visits, prescriptions, nurse visits, text messages, phone calls, billings, surgeries, and the like. Many healthcare providers believe that the sequence of these encounters can be used to predict future diagnoses and/or other measures of patient health. However, there are four main problems in using encounter data for predictive modeling. First, a patient may have many thousands of encounters with a healthcare system, making analyses computationally intensive. Second, there is an enormous variety of patient-hospital encounter sequences, especially when a patient has been involved with healthcare for many years. The large variety of sequences makes it difficult for algorithms to learn patterns that can be used for predictive purposes. Third, machine learning algorithms learn from numerical data. The encounters must be converted into a semantically consistent numerical form so that the encounter data can be used for predictive modeling. Fourth, current predictive models that use encounter sequences are extremely difficult to interpret, which makes them difficult to use in clinical settings. It would be beneficial to provide improvements in one or more of these four areas.

Predictive models are becoming more and more common in healthcare settings, but, by themselves, they do not predict treatment decisions to benefit patient health. Healthcare providers are required to interpret predictive results and other patient data in order to make treatment decisions. Because of the vast and ever-increasing number of treatment options and despite years of training and experience, healthcare providers often make suboptimal decisions, either with respect to patient health, healthcare costs, or both.

One example of a current predictive model in this art is described in a publication authored by Alvin Rajkomar et al., entitled “Scalable and Accurate Deep Learning with Electronic Health Records” (accessible at https://www.nature.com/articles/s41746-018-0029-1.pdf). The Rajkomar publication describes the converting of patient encounters into very long hash tables, and using those hash tables as training data for deep learning models. While the accuracy of these models was promising, training them was computationally intensive (taking weeks), and training could only be performed on proprietary servers using proprietary methods. Because hash tables were used as training data and deep learning techniques were used to train the models, these models were also very difficult to interpret, which made them unappealing for clinical use.

Current software systems deliver predictions about patient health without necessarily delivering the decisions that can be made to improve patient health. Testing of patient behavior modifications is limited to testing one variable at a time—for example, the variable of weight can be altered based on the hypothetical patient action of “what if the patient loses 10 pounds?” This method has shortcomings, because patient health depends on hundreds of variables, not just one, and it can be impossible to determine an optimal patient health decision based on testing of one variable at a time when the optimal solution requires multiple variable adjustments.

For example, consider running a test on a large patient population, in which a variable is adjusted to artificially lower every patient's weight by 10%. With this variable adjustment, a prediction can be made of how many more patients in the population would have well-controlled blood pressure. However, in a conventional predictive software system, the result of the prediction would be nearly 0%, because the system would not have any way to include other health benefits that a patient would receive from such weight loss (beyond just the change in weight itself) in the modified data.

### SUMMARY

According to an aspect of the present disclosure, a computer-implemented method of training a predictive patient health machine learning model includes configuring, by a computing device, patient-healthcare system encounter data formed by sets of electronic medical records of each patient of a patient population associated with the subject health parameter as a plurality of directed graphs; quantifying parameters of each directed graph of the plurality of directed graphs to generate graphed health data; and training a health predictor to predict a future state of a subject health parameter using baseline health data, the baseline health data including sets of features, wherein the sets of features include sets of record features that are extracted from the sets of electronic medical records and include the quantified parameters of the graphed health data, wherein the health predictor is a machine learning model.

According to an additional or alternative aspect of the present disclosure, a method of generating treatment information regarding future patient health includes extracting a first set of features from patient-healthcare system encounter data formed by sets of electronic medical records of each patient of a patient population associated with a subject health parameter; configuring the patient-healthcare system encounter data as a plurality of directed graphs; quantifying parameters of each directed graph of the plurality of directed graphs to form a second set of features; labeling each feature of the first set of features and the second set of features as corresponding to a future outcome with respect to the subject health parameter to generate baseline health data; training a machine learning model to predict a future status of the subject health parameter based on the baseline health data, wherein the machine learning model is implemented on a health evaluator having memory and control circuitry; receiving, by the health evaluator, pertinent health data regarding a subject patient; analyzing, by the machine learning model, the pertinent health data to generate predictive heath data representative of an expected patient condition based on the pertinent health data; and outputting, by the health evaluator, the predictive health data for the subject patient.

According to another additional or alternative aspect of the present disclosure, a method of predicting future patient health includes receiving, by a machine learning model trained to identify a future parameter status of a subject health parameter based on the baseline health data, pertinent health data regarding a patient, wherein the machine learning model is implemented on a health evaluator having memory and control circuitry, and wherein the baseline health data is generated based on sets of features extracted from sets of electronic medical records of each patient of a patient population associated with the subject health parameter; analyzing the pertinent health data, the machine learning model, to generate predictive health data regarding an expected patient condition; generating, by the health evaluator, treatment information for the patient based on one or more sets of the predictive health data; and outputting, by the health evaluator, the treatment information.

According to yet another additional or alternative aspect of the present disclosure, a method of generating predictive future health information includes (i) receiving pertinent health data, including vital signs and electronic medical records, associated with a subject patient; (ii) setting initial design variables representing modifiable risk factors associated with the subject patient; (iii) predicting laboratory results associated with the patient, by a machine learning model trained on baseline health data that is generated based on sets of electronic medical records of patients in a patient population, based on the received pertinent health data and the initial design variables; (iv) generating predictive health data for the subject patient, by the machine learning model, based on the predicted laboratory results and the pertinent health data; (v) comparing, by a health evaluator having control circuitry and memory and configured to implement the machine learning model, the predictive health data and control data to determine whether the predictive health data corresponds with desired patient health; and (vi) outputting the predictive health data as treatment information based on the health evaluator determining that the predictive health data corresponds with desired patient health.

### BRIEF DESCRIPTION OF THE DRAWINGS

FIG. 1 is a block diagram illustrating a health evaluator.

FIG. 2 is a flowchart illustrating a method of health simulation and optimization.

FIG. 3 is a flowchart illustrating a method of generating treatment information predictive of patient health.

FIG. 4 is a flowchart illustrating a method of training a health prediction machine learning model.

FIG. 5 is a diagram illustrating an example of a directed graph for mapping patient-hospital encounters.

### DETAILED DESCRIPTION

The present disclosure combines machine learning techniques with optimization techniques in a software system for providing individualized and predictive patient healthcare. Patient electronic medical records (EMRs) are used to train a number (for example, tens, hundreds, or thousands) of machine learning models to predict various patient health metrics, such as lab results and patient responses to various medications. These models are used by software that simulates the patient's response to changes in both the patient's treatment (e.g., nurse visits, different medications, etc.) and the actions the patient can take to improve health (e.g., losing weight, stop smoking, etc.). Modifiable patient risk factors and testing and simulation across a range of possible healthcare actions or interventions are used to design an enhanced (e.g., in some cases, optimal) set of actions that can be performed to improve patient health. Such actions can be delivered to both the healthcare provider and the patient, such as via mobile applications among other options.

The medical diagnosis and treatment process may be modeled as an equation, in which the sum of vital signs and other measurable patient parameters, lab results, and medical professional observations provides a candidate medical diagnosis, and the candidate diagnosis is correlated to a set of treatments and patient actions that are recommended to improve patient health. The treatments and patient actions may include one or more of medications, behavioral changes, or interventions, for example. This equation may be generally represented as follows:

Vitals+Labs+Observations=>Diagnoses=>Treatments & Actions

The medical diagnosis and treatment equation may further include other factors that influence the ultimately recommended treatments and/or actions, such as patient social determinants or genetics, for example. Thus, where information on such other factors is present, that information can be included in the equation, either in the sum of factors that lead to a candidate medical diagnosis, or in the correlation of the candidate medical diagnosis to the recommended treatments and patient actions.

In one example, the software system of the present disclosure can analyze a patient's current vital signs along with prior lab results, prior medical diagnoses and prior medications (i.e., patient historical information), and make an accurate prediction of what the results of a new/current lab test will be, based on the result of one or more predictive models applied to those inputs. With those predicted lab results, the software system can provide an accurate candidate medical diagnosis, and predict what treatment, patient behavior, and/or interventions will result in an improved patient health outcome. By using the predictive model to predict how patient behavior, medications, and interventions affect future vital signs and lab results, in order to predict future diagnoses, beneficial patient behavioral changes, medications and interventions can be identified that will improve patient health (e.g., decrease the number of comorbidities of the patient, decrease medication side effects, decrease the number of medications, decrease costs, etc.).

The present disclosure further provides a software solution that maps patient-hospital encounters by directed graphs. The present disclosure further provides a health prediction machine learning model that is trained on baseline data formed, at least in part, based on baseline training data configured as directed graphs. Properties of the directed graphs are quantified and incorporated into machine learning data models for the development of predictive analytics. In a specific example, directed graphs are built based on patient EMRs. The EMRs can be considered to form some or all of the patient-healthcare system encounter data that is used to build the directed graphs. The directed graphs are utilized to generate the baseline data to train the machine learning data models. The machine learning models are configured to predict patient outcomes. The machine learning models are based on real world patient-healthcare system encounters (i.e., the EMRs) that provide strong correlation between actions and outcomes. Such machine learning models can quickly and efficiently generate predictive health data for patients.

The directed graphs representing patient-hospital encounters are subjected to perturbations to generate additional training data for the health prediction model. The perturbations can be in one or both of the time and the resolution of the directed graph. Utilizing the perturbations in the directed graphs facilitates the machine learning algorithms learning both gross and fine patterns predictive of patient health. Perturbations in time and resolution facilitate recognition of patterns that may not otherwise be apparent.

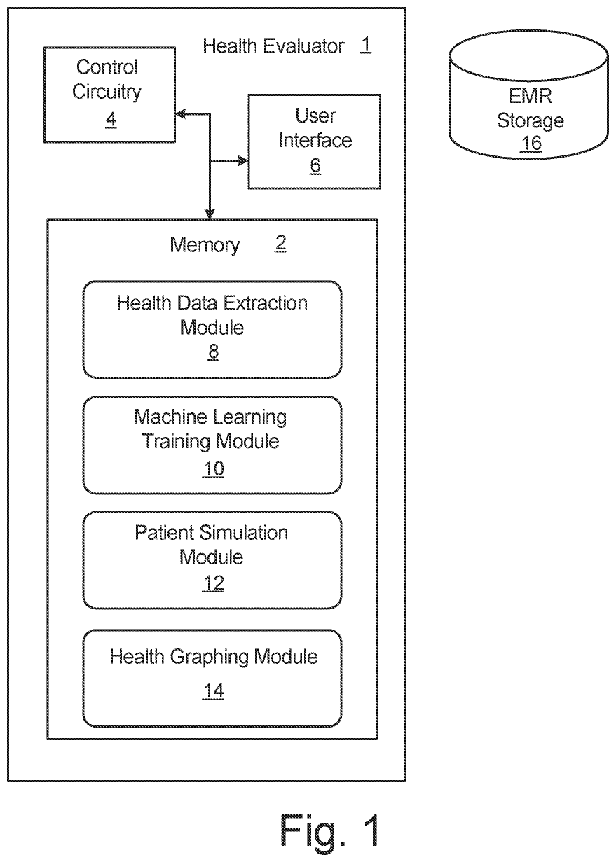

FIG. 1 is a block diagram illustrating health evaluator 1. Health evaluator 1 includes memory 2, control circuitry 4, and user interface 6. Health evaluator 1, which can also be referred to as a computing device, can be configured to implement one or more machine learning models trained on baseline health data to generate and output treatment actions identified by the health evaluator 1 as likely to enhance patient treatment, such as by improving patient health (e.g., decreasing comorbidities, normalizing lab results, etc.) and/or decreasing healthcare costs (e.g., out-of-pocket costs to the patient, minimizing resource consumption such as consumption of nonrenewable helium). The health evaluator 1 can determine the set of actions that patients and providers can take to attain an enhanced (e.g., optimal, in some examples) set of treatment actions. The baseline health data can be generated based on EMRs of patients. The memory 2 can store health data extraction module 8, machine learning training module 10, patient simulation module 12, and health graphing module 14.

The health evaluator 1 is configured to generate data and information regarding predictive patient health for a patient. The predictive health data can include sets of health information regarding predicted lab results, predicted diagnoses, and/or proposed treatment actions, among other predictive health options. The health evaluator 1 is configured to store software, implement functionality, and/or process instructions. The health evaluator 1 can be of any suitable configuration for gathering data, processing data, etc. The health evaluator 1 can receive inputs, provide outputs, generate predictive health data based on pertinent health data, and output information regarding predictive future health of the patient and/or best actions for enhancing (e.g., optimizing) health of the patient. The health evaluator 1 can be configured to receive inputs and/or provide outputs via user interface 6. The health evaluator 1 can include hardware, firmware, and/or stored software. The health evaluator 1 can be entirely or partially mounted on one or more circuit boards.

The health evaluator 1 can be a discrete assembly or be formed by one or more devices capable of individually or collectively implementing functionalities and generating and outputting data as discussed herein. The health evaluator 1 can be considered to form a single computing device even when distributed across multiple component devices. The health evaluator 1 is configured to perform any of the functions attributed herein to the health evaluator 1, including receiving an output from any source referenced herein, detecting any condition or event referenced herein, and generating and providing data and information as referenced herein. The health evaluator 1 can be of any type suitable for operating in accordance with the techniques described herein. In some examples, the health evaluator 1 can be implemented as a plurality of discrete circuitry subassemblies. In some examples, the health evaluator 1 can include or be implemented at least in part as a smartphone or tablet, among other options. In some examples, the health evaluator 1 and/or user interface 6 can include and/or be implemented as downloadable software in the form of a mobile application. The mobile application can be implemented on a computing device, such as a personal computer, tablet, or smartphone, among other suitable devices.

Control circuitry 4, in one example, is configured to implement functionality and/or process instructions. For example, the control circuitry 4 can be capable of processing instructions stored in the memory 2. Examples of control circuitry 4 can include one or more of a processor, a microprocessor, a controller, a digital signal processor (DSP), an application specific integrated circuit (ASIC), a field-programmable gate array (FPGA), or other equivalent discrete or integrated logic circuitry. The control circuitry 4 can be entirely or partially mounted on one or more circuit boards.

The memory 2 of the health evaluator 1 can be configured to store data and information before, during, and/or after operation. The memory 2, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, the memory 2 is a temporary memory, meaning that a primary purpose of the memory 2 is not long-term storage. The memory 2, in some examples, is described as volatile memory, meaning that the memory 2 does not maintain stored contents when power to the health evaluator 1 is turned off. Examples of volatile memories can include random access memories (RAM), dynamic random access memories (DRAM), static random access memories (SRAM), and other forms of volatile memories. In some examples, the memory 2 is used to store program instructions for execution by the control circuitry 4. The memory 2, in one example, is used by software or applications running on the health evaluator 1 (e.g., by a health prediction machine learning model) to temporarily store information during program execution.

The memory 2, in some examples, also includes one or more computer-readable storage media. The memory can be configured to store larger amounts of information than volatile memory. The memory 2 can further be configured for long-term storage of information. In some examples, the memory 2 includes non-volatile storage elements. Examples of such non-volatile storage elements can include magnetic hard discs, optical discs, flash memories, or forms of electrically programmable memories (EPROM) or electrically erasable and programmable (EEPROM) memories.

As illustrated in FIG. 1, the memory 2 can be configured to include health data extraction module 8, machine learning training module 10, patient simulation module 12, and health graphing module 14. Modules 8, 10, 12, 14 can take the form of computer-readable instructions that, when executed by control circuitry 4, cause the health evaluator 1 to implement functionality attributed herein to modules 8, 10, 12, 14. Though the example of FIG. 1 is described with respect to separate modules 8, 10, 12, 14, it is understood that the techniques described herein with respect to such modules 8, 10, 12, 14 can be implemented in a single module or multiple modules (e.g., two, three, four, etc.) that distribute functionality attributed herein to modules 8, 10, 12, 14 among the multiple modules. In general, memory 2 can store computer-readable instructions that, when executed by control circuitry 4, cause health evaluator 1 to operate in accordance with techniques described herein.

The user interface 6 of the health evaluator 1 can be configured as an input and/or output device. For example, the user interface 6 can be configured to receive inputs from a data source and/or provide outputs regarding patient health. Examples of the user interface 6 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other type of device for facilitating input and/or output of information in a form understandable to users and/or machines.

In operation, the health evaluator 1 executes the modules 8, 10, 12, 14 to generate pertinent health data for a patient, analyze the pertinent health data by a machine learning model to generate predictive health information regarding a future health status of the patient.

The health evaluator 1 is configured to generate predictive health data for a patient based on pertinent health data regarding the patient. The pertinent health data is provided to the health evaluator 1. For example, the health evaluator 1 can execute the health data extraction module 8 to generate the pertinent health data. Electronic medical records of the subject patient can be extracted from electronic medical record (EMR) storage 16 and provided to health evaluator 1. The EMR storage 16 can be a computer-readable database configured to store data regarding electronic medical records of one or more patients. The pertinent health data can be generated based on the EMRs of the patient received from the EMR storage 16.

The pertinent health data can include patient data, which is data about the specific patient (e.g., age, weight, height, ethnicity, race, economic status, lifestyle factors such as smoking and drinking, comorbidities, trends regarding patient health (increasing or decreasing blood pressure, lab results, etc.), etc.); treatment data, which is data regarding treatment actions for the patient (e.g., one or more of lifestyle changes, additional or different medications, one or more procedures, frequency of follow ups, etc.); control data, which is information regarding constraints on future treatment; among other input options. The pertinent health data includes various types of information specific to the patient, such as information regarding current and past morbidities, lifestyle factors (e.g., tobacco use, alcohol use, frequency of exercise, etc.), lab results (e.g., results of blood tests, urine tests, etc.), vital signs (e.g., body temperature, pulse rate, respiration rate, blood pressure etc.), blood oxygen level, height, weight, profession, marital status, age, sex, race, household factors (e.g., number of persons in the household, location such as zip code, etc.), treatment tolerance information (e.g., the patient is particularly susceptible to nausea, the patient cannot have blurred vision, a minimum or maximum count of prescriptions, etc.), among other data regarding the patient.

The health evaluator 1 is configured to implement one or more health prediction machine learning models to generate predictive data regarding future health of a subject patient. The health evaluator 1 can execute the machine learning training module 10 to train the one or more machine learning models. The trained machine learning models can be stored in the memory 2. The health prediction models are trained to predict the future status of one or more factors regarding the health of a patient based on pertinent health data regarding the patient.

The health evaluator 1 can execute the machine learning training module 10 to train the health prediction models. The health prediction models are trained on baseline health data that is generated based on sets of EMRs for each patient in a patient population. For example, the EMRs can be extracted from the EMR storage 16. The health data extraction module 8 can extract various parameters from the EMRs to generate the baseline health data. The patient population is formed by patients associated with one or more subject health parameters. For example, the subject health parameter can be a morbidity (e.g., hypertension, diabetes mellitus, congestive heart failure, etc.) and the health prediction models can be trained to determine whether the morbidity will or will not be successfully treated (e.g., clinically controlled). It is understood that the subject health parameter can be any single parameter or combination of parameters regarding patient health as desired, such as one or more lab results, weight, BMI, morbidity status, etc. The patient population is a statistically significant sample size of the population. In some examples, the patient population can be each patient within a healthcare system (e.g., hospital, clinic, networks of providers, etc.) associated with the subject health parameter. For example, a patient can be associated with the subject health parameter by being diagnosed with a subject morbidity, receiving data for a subject lab result, etc. The health data regarding the patients forming the patient population is taken over a sampling period, such as one year, two years, five years, or any other desired sampling period.

The population size is large enough to support the development of predictive health analytics. In some examples, the population size can be 10,000 or more patients, 50,000 or more patients, 100,000 or more patients, 200,000 or more patients, 400,000 or more patients, 600,000 or more patients, etc. In some examples, the population size can be ten times or more larger than the number of subject health parameters in the sets of EMRs, providing at least a 10:1 ratio of population size to subject parameters. The sets of EMRs to subject parameter ratio can be 20:1, 30:1, 40:1, 100:1, or any other desired ratio suitable for generating statistically significant results. The baseline health data includes information regarding encounters between the patients and the healthcare system, which includes one or more encounters per patient. As such, the baseline health data can include multiple sets of EMRs providing information from multiple encounters for each patient. For example, a ratio of encounters to patients in the patient population can be 1.5:1, 2:1, 3:1, 4:1, or more.

Health evaluator 1 can, by the health prediction models, identify health factors for a patient that are anticipated to be effective at improving the patient's health. For example, the health evaluator 1 can execute the patient simulation module 12 to simulate patient health based on the pertinent health data. The health evaluator 1 can identify modifiable factors and, in some examples, simulate changes to those modifiable factors to identify the factor or factors that can be modified to effectively improve patient health (e.g., by minimizing the number of prescriptions, minimizing number of morbidities, stabilizing certain factors (e.g., blood pressure), normalizing certain lab results, etc.). The health evaluator 1 can execute the patient simulation module 12 to identify one or more factors for modification to improve the health of the patient and can generate modified treatment actions for the patient, based on the pertinent health data.

The one or more health predictor models are trained based on baseline health data that is generated based on patient data for patients forming a patient population associated with the health parameters. The patient data can include information extracted from sets of EMRs of the patients forming the patient population. The sets of EMRs provide information regarding health factors of the patients in the patient population.

Sets of features are extracted from the sets of EMRs. The sets of features extracted from the EMRs, either directly or derived, can be referred to as record features as such feature are extracted from electronic medical records. Each set of features is formed based on a patient within the patient population. Each set of features includes pertinent health data regarding a patient, such as information regarding current and past morbidities, lifestyle factors (e.g., tobacco use, alcohol use, frequency of exercise, etc.), lab results (e.g., results of blood tests, urine tests, etc.), vital signs (e.g., body temperature, pulse rate, respiration rate, blood pressure etc.), blood oxygen level, height, weight, profession, marital status, age, sex, race, demographic information (e.g., number of persons in the household, location such as zip code, first language, employment status, etc.), health trends (e.g., test levels associated with the subject morbidity trending up or down, etc.), among other patient information. Each set of features includes data regarding the treatments utilized for the patient to treat the one or more morbidities of the patient. In some examples, the treatment data can include information regarding the types and dosages of medications prescribed. In some examples, the treatment data includes the costs associated with various treatment actions, such as out-of-pocket costs for drugs, physical therapy, procedures, etc. It is understood that the treatment data can include any desired information regarding the treatments utilized by the patient. Each set of features can include tens, hundreds, or thousands of features. For example, each set of features can include greater than or equal to five hundred features, greater than or equal to seven hundred features, greater than or equal to nine hundred features, among other options. The set of features for each patient is formed from data that is taken directly from the EMRs and that is derived from the EMRs.

The various features forming each set of features can be extracted directly from the EMRs and/or derived from information contained in the EMRs. For example, lab results, height, weight, body mass index (BMI), etc. can be taken directly from the EMRs to form some of the features in each set of features. Other ones of the features can be derived from the information contained in the EMRs.

Health trend data can be derived from the information contained in the EMRs. For example, trends in weight, trends in BMI, trends in lab results, trends in systolic blood pressure, trends in diastolic blood pressure, etc. can be determined from the information in the EMRs and utilized to form features within each set of features. The health trend data can include information regarding medications that is derived from the EMRs. For example, trends in the number of medications, such as increasing or decreasing numbers of medication, increasing or decreasing dosages, etc. can be derived from the EMRs.

Health minimum and maximum data can be derived from the information contained in the EMRs. For example, the minimum or maximum values for various health parameters, such as blood pressure, weight, BMI, lab results, etc., can be derived from the EMRs to form features. The minimum and maximum values can be taken within a certain time period, such as within a time period before treatment began, a time period after treatment began, etc.

Demographic data regarding a patient can be derived from the information contained in the EMRs. For example, the patient's residence location can be determined from the EMRs and demographic data, such as median income, average household size, etc., can be determined based on census data for that residence location.

Encounter data regarding patient encounters with the healthcare system can be derived from the EMRs. For example, the number and/or frequency and/or type of encounter between the patient and healthcare system can be derived from the EMRs. The encounter data can provide information on how a patient interacts with the healthcare system, such as by phone, in person, etc.

The sets of features are labeled to train the health prediction models. The sets of features can be labeled in any desired manner based on the subject health parameter, such as whether a subject morbidity is or is not clinically controlled; whether a lab result is within a normal range, high, or low; etc. The outcomes can be associated with a temporal threshold for the subject health parameter. The temporal threshold can be associated with the treatment being initiated, such as when a treatment plan is initially prescribed. In such an example, the temporal threshold can be taken from when treatment is initiated for that patient. In some examples, the same set of features is associated with different outcomes depending on the temporal threshold. For example, the sets of EMRs of a first patient of the patient population can indicate that the subject lab results entered a normal range at a time fifteen months after treatment began. The set of features extracted from the sets of EMRs of that first patient are associated with a positive outcome for a temporal threshold of at least fifteen months, while that same set of features extracted from the same sets of EMRs of that same first patient are associated with a negative outcome for temporal thresholds less than fifteen months.

The sets of features provide baseline health data for training the health predictor model. Each set of features can be configured as a row of data with the individual features formed in columns. As such, the baseline health data can be configured as a table with each row of data representing a set of features and each column including a feature of that set of features. The health predictor model utilizes machine learning to analyze, understand, and/or respond to patient data. By analyzing a selection of baseline health data, the treatment machine learning model (e.g., decision trees, boosted decision trees, deep learning algorithms, linear regression models, neural networks, confidence assessments, fuzzy logic, among other options) can be trained to recognize, classify, and react to pertinent health data. The machine learning algorithms are computationally complicated and difficult to implement at scale. In some examples, the machine learning model can comprise majority voting among multiple classification machine learning models that together can be considered to form the health predictor machine learning model, as discussed in more detail below. The health predictor model can be configured as an ensemble model configured to generate a prediction based on the multiple predictions output by the classification models.

In some examples, the health evaluator 1 can execute the health graphing module 14 and configure the baseline health data as directed graphs. The health of a patient can be quantified at various points that can be represented in the directed graph. For example, the health evaluator 1 can determine relative severities of diagnoses, relative severities of conditions, relative importance of lab results, relative severities of drug interactions, etc. based on the baseline health data. In some examples, encounter data can be quantified and provided to the health evaluator, such as by the user quantifying the data.

The health evaluator 1 can execute the machine learning training module 10 to train the one or more health predictor models based on baseline health data. An example of training a health predictor model is discussed in additional detail. The baseline health data is split into a first dataset and a second dataset for training the treatment machine learning model. The first dataset can be referred to as training data and the second data set can be referred to as testing data. Each set of features forming the baseline health data are placed in one of the first dataset and the second dataset. The sets of features are randomly assigned to the two datasets such that the sets of features in each of the first dataset and the second dataset are representative of the patient population. As such, each of the first dataset and the second dataset are representative of the patient population as a whole. The first dataset includes more sets of features than the second dataset. In one example, the first dataset is formed by two thirds of the baseline health data and the second dataset is formed by one third of the baseline health data. The first dataset can be formed by 60%, 70%, 80% or another majority percentage of the baseline health data. For example, the first dataset can include a majority number of the rows of the baseline health data. The second dataset is formed by the remainder of the baseline health data, such as 40%, 30%, 20%, or another minority percentage of the baseline health data. For example, the second dataset can include a minority number of the rows of the baseline health data.

Training of the health predictor model includes an initial training based on the first dataset and testing of that initially trained model based on the second dataset. The health predictor model is initially trained on the first dataset. The labeled sets of features of the first dataset are provided to the health predictor model and the machine learning algorithm is configured to determine a fit to arrive at the labeled conclusion based on the set of features (e.g., to determine whether a subject morbidity is or is not controlled; determine a predicted lab result; etc.). The health predictor model undergoes supervised learning because the sets of features are labeled with the correct outcome during the training phase of the machine learning model.

The health predictor model can be an ensemble model configured to generate a prediction based on predictions from multiple classification models. The classification models are individual machine learning models (e.g., decision trees, linear regression, etc.) that are individually trained on the baseline health data to generate a prediction regarding patient health. The multiple classification models together form the health predictor model. The health predictor model can generate a final prediction based on individual predictions made by multiple classification models forming the health predictor model. In a specific example, the health predictor model is formed based on gradient boosted decision trees. The health predictor model can be based on parallel decision tree boosting. In one example, the health predictor model utilizes the XGBoost algorithm during training of the health predictor model.

In a specific example, the health predictor model is provided with the first dataset. The machine learning algorithm is configured to generate decision trees based on the first dataset. Each decision tree can be considered to form a classification model of the health predictor model. In one example, each decision tree is provided with a factor subset that defines the factors that that decision tree considers. For example, if each set of factors includes nine hundred factors, then a first factor subset may contain five hundred of those factors, or another number of those factors. The machine learning algorithm generates a first decision tree based on that first factor subset. A second factor subset may contain another five hundred of those factors, and the factors forming the second factor subset can overlap with one or more of the factors forming the second factor subset. The decision tree is configured such that the decision tree considers only those factors within the factor subset applied to that decision tree. The decision tree can disregard other factors not present within the factor subset of that decision tree. The overlap between the various factor subsets can vary. In some examples, the factor subsets may not overlap. In some examples, the factor subsets may substantially overlap, such as by having 90% or more commonality between factors.

Each decision tree is formed as a series of nodes with bifurcating branches that extend from each node. A prediction as to a health parameter (e.g., whether a morbidity is controlled, the level of a lab result, etc.) is made once a terminal node is reached. Each node is configured as if-then-else statements where a first pathway from the node is followed if all of the rules within the node are satisfied and a second pathway from the node is followed if less than all of the rules of the node are satisfied. Each node can be based on one or multiple of the factors forming the factor subset that that decision tree considers.

Parameters of the decision trees are defined prior to the machine learning algorithm constructing the decision trees. Such a process can be referred to as hyperparameter tuning. Hyperparameter tuning involves choosing a set of optimal hyperparameters for the machine learning algorithm prior to the learning process beginning. For example, the hyperparameters regarding a decision tree can define the number of layers forming the tree or the width of the tree. The machine learning algorithm generates the decision trees based on the first dataset and the hyperparameters. While the hyperparameters are defined, the values of other parameters, such as the weights assigned to various factors (e.g., in a linear regression model) or the weights assigned to various decision trees (e.g., in a gradient boosted decision trees model), are learned by the machine learning algorithm during the training of the machine learning model.

Each decision tree is iteratively constructed by the machine learning algorithm based on the first dataset and the factor subset for that decision tree. For example, the first decision tree based on the first factor subset can be constructed based on possible decisions for each patient in the first dataset and based on the first factor subset. Each decision tree is formed in a similar manner, but based on different factor subsets. For each decision tree, the machine learning algorithm generates the nodes and the if-then-else statements associated with each node based on the first dataset and the factor subset for that decision tree. It is understood that tens, hundreds, or thousands of decision trees can be generated based on the first dataset.

The multiple decision trees are generated based on the labeled first dataset. After generation, each decision tree is tested based on the second dataset. During testing, the decision trees are provided the unlabeled sets of factors forming the second dataset and the decision trees generate predictions regarding the subject health parameter. The treatment machine learning model has not been exposed to the second dataset prior to the testing stage. The testing stage provides information on the accuracy of the predictions generated by each decision tree of the health predictor model as the outcome (e.g., the actual level of the lab result) is already known for each patient forming the second dataset. The predictive accuracy of each decision tree is determined based on the performance of each decision tree at predicting the subject health parameter across the second dataset.

The decision trees are weighted based on the predictive accuracy determined from executing each decision tree on the second dataset. A weighting factor is generated for and applied to each decision tree based on the predictive accuracy of each decision tree. More accurate ones of the decision trees, which are the decision trees that had a higher accuracy rate for simulations based on the second dataset, will be assigned a higher weighting factor. More weight is given to the result output by decision trees having higher weighting factors. Less accurate ones of the decision trees will be assigned a lower weighting factor. Less weight is given to the result output by decision trees having lower weighting factors. The weighting factor is based on the accuracy of the prediction for that decision tree across the patient population, as determined based on the predicted outcomes for the second dataset.

The weighted decision trees form the trained health predictor model. During execution of the health predictor model, each decision tree of the health predictor model generates a prediction as to a future status of the subject parameter based on the subset of factors that each decision tree considers. Each decision tree is configured to determine a probability, such as that a lab result will be in a normal range. For example, each decision tree can output the probability as a value between zero and one, with prediction values greater than or equal to 0.5 indicating agreement (e.g., that the lab result will be in the normal range) and prediction values less than 0.5 indicating disagreement (e.g., that the lab result will not be in the normal range). The prediction values of all of the decision trees forming the health predictor model are normalized such that the health predictor model generates a prediction value between zero and one. The health predictor model generates the prediction regarding future health based on the overall prediction value formed from the weighted outputs from the classifiers.

While a specific training example regarding boosted decision trees is discussed, it is understood that the health predictor model can be trained based on any desired machine learning algorithm, such as linear regression algorithms or deep learning algorithms. In some examples, the health predictor model can be trained based on a linear regression algorithm. In such an example, the machine learning algorithm is configured to learn weights applied to each of the factors to minimize an error between the predicted value and a true value. The linear regression equation is shown in Equation 1.

Y=w1X1+w2X2+w3X3 . . . +wnXn+B (Equation 1)

Y is the output variable that is being predicted by the model, such as whether the subject morbidity will or will not be clinically controlled. Each X is a different factor from the set of factors that form the patient data. Each w is a weighting factor for X associated with that weighting factor, and the weighting factors are learned by the treatment machine learning model during training. B is a residual error factor that is also determined by the machine learning algorithm during training. The linear regression model is initially trained based on the information in the first dataset.

In some examples, the health predictor model can be an ensemble model based on multiple linear regression models. Such an ensemble model can be configured similar to the health predictor model based on multiple decision trees. For example, each linear regression model can be configured to make a health prediction based on less than all of the features forming the sets of features. The linear regression models can be assigned weights based on the predictive accuracy of the linear regression models for the second dataset. The health predictor model can generate the health prediction based on a normalized output from each of the multiple linear regression models.

In some examples, multiple sets of baseline health data are generated based on different temporal thresholds and those multiple sets of baseline health data are used to train multiple health predictor models based on the various temporal thresholds. For example, a first health predictor model can be trained based on baseline health data associated with a six month temporal threshold, a second health predictor model can be trained based on baseline health data associated with a twelve month temporal threshold, etc.

The health predictor model can identify various ones of the factors as positively corresponding with various outcomes. For example, the data generated by the health predictor model can indicate that trend in weight, age, and number of medications have a high correlation with a subject morbidity. The health predictor model can further identify various ones of the factors are negatively corresponding with various outcomes. For example, the data generated by the health predictor model can indicate that trend in BMI,

Health evaluator 1 can execute the patient simulation module to generate predictive health data for a patient based on pertinent health data regarding that patient. In some examples, the health evaluator 1 can generate information regarding the future health of the patient, such as whether a morbidity will or will not be controlled. In some examples, the health evaluator 1 can generate information regarding treatment options for a patient given desired health outcomes. For example, the health evaluator 1 can be configured to generate information regarding treatments that are predicted to achieve the desired health outcome, such as by normalizing a lab result.

Risk factors of the patient can be modified to generate modified health data for the patient. The health evaluator can execute the patient simulation module 12 to generate the modified health data for the patient. The health evaluator 1 can generate predictive health data based on the modified health data to provide an indication of predicted health statuses for a patient having the modified health data. The health evaluator 1 can be configured to generate treatment data associated with the modified health data. For example, the health evaluator 1 can execute the patient simulation module 12 to generate customized treatment plans for the patient that correspond with the patient achieving the health parameters in the modified health data. The customized treatment plans correspond to the actual condition of the patient, as set forth in the pertinent health data, which allows for the generation of personalized healthcare plans for that patient.

The health evaluator 1 facilitates generating treatment information specific to a patient that may not be apparent when otherwise analyzed. The health evaluator 1 can determine that certain actions may be more helpful for a first patient than a second patient and can generate treatment information based on those specific actions. For example, a certain lab test level may be high for a patient, but the health evaluator 1 can determine based on the pertinent health data that it may not be most beneficial to attempt to control that specific lab level for persons of a certain age, but that it may be highly beneficial for others. In one example, the trained health prediction models can determine, based on the pertinent health data, that in-home nurse visits are more beneficial for a first patient (e.g., an 83 year old living alone) than for a second patient (e.g., a 26 year old living with others) and the health evaluator 1 generates sets of predictive health data in accordance with the best actions for the specific patient.

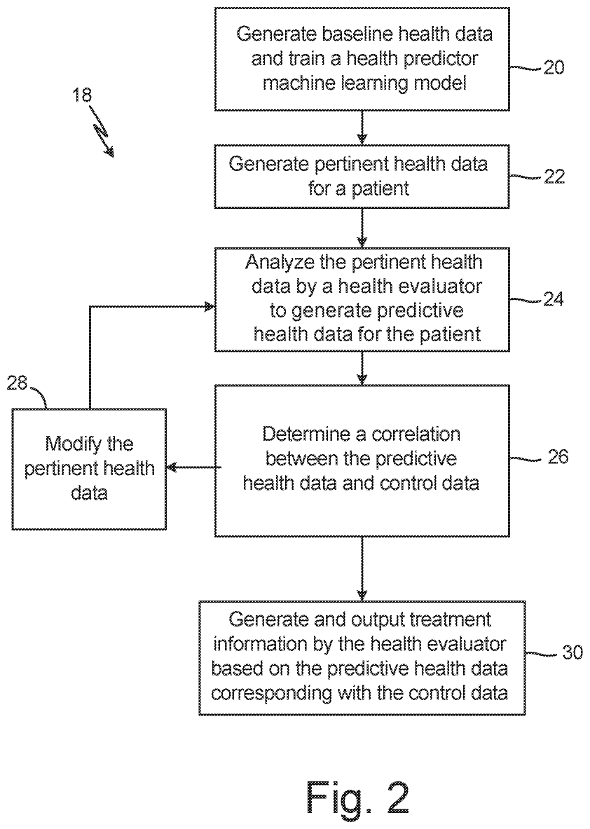

FIG. 2 is a flowchart illustrating method 18 of health simulation and optimization. In step 20, one or more health machine learning models are trained on baseline health data to generate predictive health data regarding a patient. A computing device, such as health evaluator 1, is configured to implement the health predictor models to generate the predictive health data. It is understood that individual machine learning models can be trained to generate predictive health data regarding discrete aspects of patient health (e.g., generate predictive health data regarding a single lab result (e.g., creatinine), a single morbidity (e.g., hypertension), etc.). The multiple individual machine learning models can be considered to collectively form the health predictor model. Each machine learning model of the health predictor model can be trained as discussed above with regard to FIG. 1. In some examples, the health predictor models can be trained on baseline health data configured based on directed graphs to generate and output the predictive health data.

The baseline health data is based, at least in part, on information extracted from EMRs that are taken from across many patients and encounters. The baseline health data is configured as sets of features that form portions of the baseline health data. The baseline health data is used to train the one or more machine learning models to predict patient health metrics, such as lab results and patient response to various medications, among other health metrics. The machine learning models are used by the health evaluator to simulate patient responses to changes in both treatment actions (e.g., nurse visits) and patient actions (e.g., lifestyle changes) to improve patient health.

In step 22, pertinent health data is generated and provided to the health evaluator. The pertinent health data can include the current patient data and/or treatment data regarding the patient. In some examples, control data can be generated and provided to the health evaluator. The control data can be considered to define the ultimate treatment goals for the patient. For example, the control data can define treatment constraints, which can include that a patient's diagnosis (e.g., hypertension) is to be controlled by a certain point in the future (e.g., six months, twelve months, or any desired time period); that a count of the patient's comorbidities is minimized; that treatment costs are minimized; that a count of medications is minimized; that lifestyle changes are minimized, etc. The control data can be generated based on patient goals and/or general patient health, among other options. The patient can generate at least some of the control data, such as prior to an office visit or on an intake form, among other options. In some examples, the control data can be generated via the graphical user interface (GUI) of a mobile application, which can, in some examples, be configured to implement the health evaluator 1. The control data can be pre-generated and provided to the health evaluator, such as by a user.

The pertinent health data includes various health parameters, that can also be referred to as risk factors. The risk factors can be considered to form design variables of the system. At least some of the risk factors forming the pertinent health data can be considered to be modifiable. For example, lifestyle changes can be made by the patient (e.g., nicotine cessation, increased exercise, maintaining or commencing a certain diet, etc.), the patient's lab results can be changed as a result of changes to medication and/or lifestyle changes, follow-ups with nurses and/or doctors can be scheduled or intervals between such checks can be changed, etc. Others of the risk factors can be considered to be fixed. For example, the fixed risk factors can include and/or be formed by historical data, such as historical patient data and/or historical treatment data, and can include components of the pertinent health data, such as patient age, height, race, etc. For example, historical medical records of the patient (i.e., EMRs of the patient) can be provided to the health evaluator as part of the pertinent health data and the historical health data that can be derived from such medical records is fixed. At least some of the variables forming the pertinent health data can be considered to be fixed.

Various health parameters can be identified and set as modifiable or fixed for the health evaluation conducted by the health evaluator 1. In some examples, the user, such as the patient or doctor, can categorize various health parameters as either modifiable or fixed. For example, a patient can provide personal preferences regarding the potential for modification for various ones of the risk factors, such as via an intake form or a mobile application. For example, a patient may indicate that they prefer fewer medications, are unwilling to increase exercise, are amenable to changes in diet, etc. Pertinent health data in the form of modifiable health parameters, constraints, fixed health parameters, historical health data of the patient, and/or any other information regarding the patient that has been acquired (e.g., lab results, blood pressure reading, data regarding patient lifestyle factors) is provide to the health evaluator to generate the predictive future health information for the patient.

In step 24, the health evaluator analyzes the pertinent health data to generate predictive health data regarding future health of the patient. The predictive health data can include information regarding health actions (e.g., treatment actions and/or patient actions), expected statuses for various health parameters, etc. The health evaluator is configured to implement one or more machine learning models trained on the baseline health data to generate the predictive health data regarding the patient.

The machine learning models generate the predictive health data. The machine learning models generate information regarding future health of the patient based on the pertinent health data. For example, the one or more of the machine learning models can be configured to predict the status of certain lab results at a point in the future, to predict the status of certain morbidities, etc.

One or more sets of the predictive health data can be generated and analyzed to determine improved (e.g., optimal) treatment actions for the patient. In some examples, the various sets of predictive health data can be aggregated to form treatment information for the patient. In step 26, the health evaluator determines a correlation between the predictive health data generated in step 24 and the control data to generate information regarding a correlation between the predictive health data and target outcomes for the patient. For example, if a constraint in the control data is that patient hypertension is controlled twelve months in the future, the health prediction model can be configured to identify treatment actions that correspond with controlling patient hypertension in the next twelve months. In such an example, the predictive health data generated by the health evaluator can be considered to correspond with the control data based on the predictive health data indicating control of the patient's hypertension within twelve months. Predictive health data determined to meet one or more of the requirements of the control data can be classified as correlated with desired patient health.

The health evaluator can be configured to determine the predictive health data that best corresponds with target patient health outcomes. In some examples, the health evaluator can generate multiple sets of predictive health data for the patient based on the initial set of pertinent health data. For example, the health evaluator can generate a first predictive health data based on treatments directed to a first morbidity and generate second predictive health data based on treatments directed to a second morbidity. The health evaluator can identify different aspects of the patient's health based on the different simulations, facilitating direct comparison of the treatment options.

In some examples, the health evaluator can apply ranking data to various health conditions, treatments, patient lifestyle factors, etc. The ranking data can be provided to the health evaluator as part of the baseline health data during the training of the health predictor models, can be generated by the health predictor model during simulation, can be stored in memory 2, among other options. For example, the ranking data can provide information on the relative severities of various morbidities and the health evaluator can rank the outputs forming the predictive health data based on such ranking data. The severity of each morbidity can be indicated by a ranking score associated with that morbidity.

The health evaluator can generate scored sets of predictive health data based on the outputs generated by the heath predictor model and the ranking data. For example, the health evaluator may determine that a patient is predicted to have four of seven original morbidities based on a first predictive output from the health evaluator and may determine that the same patient is predicted to have three of seven original morbidities based on a second predictive output from the health evaluator. The health evaluator can determine that the first predictive output is better associated with improved patient health based on a comparison of the morbidity ranking scores of the outputs, such as the morbidity ranking scores indicating that the four morbidities associated with the first predictive output are less severe than the three morbidities associated with the second predictive output.

In some examples, the health evaluator is configured to generate composite scores for the sets of predictive health data based on the comparison between each set of predictive health data and the control data. The composite scores provide information on the degree to which the predictive health data correlates with desired patient health. The composite scores can be generated based on one or more parameters of the control data. Comparisons of composite scores between each set of predictive health data and the control data providers information on the degree of the relationship between the predictive health data and the control data. For example, the closer the relationship between the predictive health data and the control data, the higher the composite score for that set of predictive health data. Such composite scoring information can be output to the user as at least a portion of the output from the health evaluator 1 to facilitate assigning specific health actions to a patient.

In some examples, the health evaluator can generate and assign ranking values to one or more parameters of a set of predictive health data and generate a composite score based on the ranking values. For example, the control data can be configured as tiers that the predictive health data can be categorized within to determine the degree of the relationship. In some examples, the comparison between the predictive health data and the control data can be a binary consideration, such as whether the predictive health data does or does not satisfy the conditions of the control data. In a binary consideration example, predictive health data that fails to meet at least one factor of the control data can be considered to not correspond with patient health.

In some examples, the health evaluator can be configured to analyze the predictive health data based on a correlation threshold. The correlation threshold can be based on how closely the predictive health data matches with the desired treatment outcome embodied in the control data. For example, the control data can have multiple desired outcomes (e.g., maintaining medications below a certain number, reducing the comorbidity count, etc.) and the correlation threshold can be based on satisfying one, two, three, or any desired number or percentage of the desired outcomes. In some examples, the correlation threshold can be based on a minimum composite score for the predictive health data, which can be generated by the health evaluator. Sets of predictive health data that satisfy the minimum composite score can be considered to correspond to desired patient health.

The health evaluator generates the predictive health data based on the pertinent health data for the subject patient. The predictive health data is thus personalized for the subject patient. Method 18 can be configured to modify the pertinent health data and generate predictive health data based on that modified health data. The health evaluator can analyze that modified health data to generate additional treatment information for the subject patient. For example, method 18 can proceed through the loop from step 26 to step 28 and back to step 24 to generate further sets of predictive health data. The health evaluator can be configured to generate multiple sets of predictive health data to identify various sets of predictive health data that correspond with future patient health.

The health evaluator can iteratively modify and analyze the pertinent health data to generate the additional sets of predictive health data. In step 28, the health evaluator modifies one or more health parameters of the pertinent health data to generate modified pertinent health data. The health evaluator can be configured to generate the modified pertinent health data based on the health evaluator determining that the predictive health data does not correspond with the control data, based on a minimum iteration count, among other iteration options. The health evaluator can be configured to modify any one or more of the modifiable health parameters to generate the modified pertinent health data. The health evaluator can be configured to modify one or both of the patient data (e.g., weight, BMI, lifestyle factors) and the treatment data (medications, procedures, etc.) to generate the modified pertinent health data.

The modification of the pertinent health data by the health evaluator can be constrained, such that certain parameters are modifiable to alter the pertinent health data while other parameters are fixed and unmodifiable. The modifiable parameters can be the modifiable risk factors and the fixed parameters can be the fixed risk factors. The modifiable parameters can themselves be constrained. For example, the value changes for certain of the parameters can be limited such that the value cannot be freely changed as desired, such as by upper and/or lower bounds, rate limits, etc. In a specific example, a variable relating to blood pressure can only be changed according to a rate limit over time (e.g., only 1% per month, only a threshold number of millimeters of mercury (mmHg) change per week, etc.).

The health evaluator generates additional and alternative sets of predictive health data by iteratively repeating steps 24-28. In some examples, the health evaluator can be configured to generate additional sets of predictive health data based on a threshold treatment plan count. The threshold treatment plan count, which can be considered to form a variable of the control data, can be one, two, three, five, ten, or any desired number of iterations. For example, if the threshold treatment plan count is five, then the health evaluator can iteratively generate predictive health data sets until five sets of predictive health data correlating with desired patient health are generated.

The health evaluator can introduce modifications to the modifiable risk factors to generate the modified pertinent health data. The health predictor model simulates future health of a modified subject patient, the modified subject patient having the modified pertinent health data. The health simulation provides information regarding the expected health of the subject patient if the subject patient achieved the modified risk factors. For example, the health evaluator can modify the weight and BMI of a subject patient and simulate future health based on the modified weight and BMI. The health evaluator generates predictive health data for the modified patient having the modified weight and BMI. Such predictive health data is indicative of the expected health of the subject patient if they were to adjust their weight and BMI to match the modified values. Such simulation provides specific information for that patient and allows the patient to ascertain the actual health benefits achieved by making such changes. The modified pertinent health data can include changes to both patient and treatment actions. As such, the health evaluator can generate information regarding the likelihood of successfully treating the patient across a wide variety of changes that can be made, such as different medications or dosages, among other options.

In step 30, the treatment information generated by the health evaluator is output to the user. The treatment information is based on the one or more sets of predictive health data generated for the subject patient. For example, the health evaluator can build the treatment information based on the one or more sets of predictive health data that correspond with the control data. The treatment information can be based on predictive results (i.e., generating predictive health data based on fixed initial treatment data) or based on predictive actions (i.e., generating predictive health data based on fixed control data). The health evaluator can generate and output health actions that correspond with improved patient health and/or with the patient achieving the modified health data associated with improved patient health. For example, the health evaluator can output one or more of treatment actions (e.g., prescriptions, doctor appointments, etc.) and patient actions (e.g., at least 30 minutes of exercise, diet changes, etc.). The health evaluator can identify the modified risk factors in the treatment information to provide clinical decision support to the provider and patient.

The health evaluator generates the one or more predictive health data sets for the patient based on the pertinent health data and modifications made thereto. The predictive health data can be provided to the patient and/or provider by way of the user interface. For example, a mobile application can provide the predictive health data to the patient and/or provider.

The health evaluator builds the treatment information based on the one or more predictive health data sets identified as corresponding with improved patient health. Some examples of the treatment information include multiple sets of predictive health data. For example, the health evaluator can determine that multiple sets of the predictive health data correspond with desired patient health. The multiple sets of corresponding predictive health data can be stored in the memory 2 of the health evaluator 1 while the health evaluator 1 iteratively generates the predictive health data. The health evaluator can build the treatment information based on each of the sets of correlated predictive health data.

In some examples, the health evaluator is configured to generate the treatment information based only on sets of predictive health data that meet all constraints of the control data. In some examples, the health evaluator can generate the treatment information based on the composite scores or degree of relationship for the predictive health data sets. For example, multiple sets of predictive health data may satisfy the control data constraints of effectively treating (e.g., curing or otherwise controlling) two of three comorbidities, minimizing a number or extent of lifestyle changes, and minimizing the number of prescriptions. The health evaluator can be configured to output a list of one or more of the sets of predictive health data that satisfy the control data. For example, a first treatment plan having three prescriptions, requiring increased exercise, and effectively treating two comorbidities can have a first composite optimization score; a second treatment plan having one prescription, requiring increased exercise and change in diet, and effectively treating one comorbidity can have a second composite optimization score; and a third treatment plan having six prescriptions, no lifestyle changes, and effectively treating two of three comorbidities can be assigned a third composite optimization score.

In some examples, the health evaluator can be configured to generate a ranked list of the sets of predictive health data based on the composite scores and can build the treatment information based on the ranked predictive health data sets. The health evaluator can rank the predictive health data sets based on treatment factors, such as one or more of the severity of the remaining one or more comorbidities, the number of medications prescribed and effects of the medications and/or combinations of the medications, lifestyle changes required by the patient, cost, among other ranking criteria. In some examples, the treatment factors can include rankings within categories. For example, comorbidities can be ranked relative to each other based on the relative severities of the comorbidities, patient input (e.g., perhaps the patient would prefer insomnia to fatigue as a potential side-effect), among other ranking factors. In one example, a parameter of the control data can be to minimize the number of prescriptions and the predictive health data sets can be ranked based on the number of prescriptions from fewest to most. In such an example, the predictive health data sets having fewer prescriptions have a stronger relationship or correlation with the control data than those predictive health data sets that include more prescriptions.

The treatment information generated and output by the health evaluator can be used to determine best treatment actions for a patient. The health evaluator and method 10 provide significant advantages. The health evaluator provides an individualized set of treatments for a patient depending on that specific patient and based on the real world EMRs. Training the health evaluator based on EMRs facilitates generating treatment information for particular patients depending on the specific patient data due to the actions, reactions, and other data available in EMRs. The EMRs provide robust data that facilitates the health evaluator learning patterns across varieties of patient data and

The health evaluator facilitates customized treatment plans based on the patient data for a specific patient. The customized treatment plans correspond to the actual condition of the patient, as set forth in the patient data, which allows for the generation of personalized healthcare plans for that patient. The health evaluator facilitates generating treatment information specific to a patient that may not be apparent when otherwise analyzed. The health evaluator can determine that certain treatments may be more helpful for a first patient than a second patient and can generate treatment information based on those specific actions. For example, a certain lab test level may be high for the patient, but the health evaluator can determine based on simulations by the health predictor model based on the pertinent health data that it may not be beneficial to attempt to control that specific lab level for certain persons but that it may be highly beneficial for others. For example, the health predictor models can learn from the baseline health data that certain health actions are more or less related to desired outcomes. The health evaluator generating patient-specific treatment options facilitates varying treatments for different patients for the same condition. For example, the health evaluator may determine, based on the pertinent health data, that in-home nurse visits are more beneficial for a first patient (e.g., an 83 year old living alone) than for a second patient (e.g., a 26 year old living with others) and the health evaluator generates sets of predictive health data in accord with best actions for the specific patient.

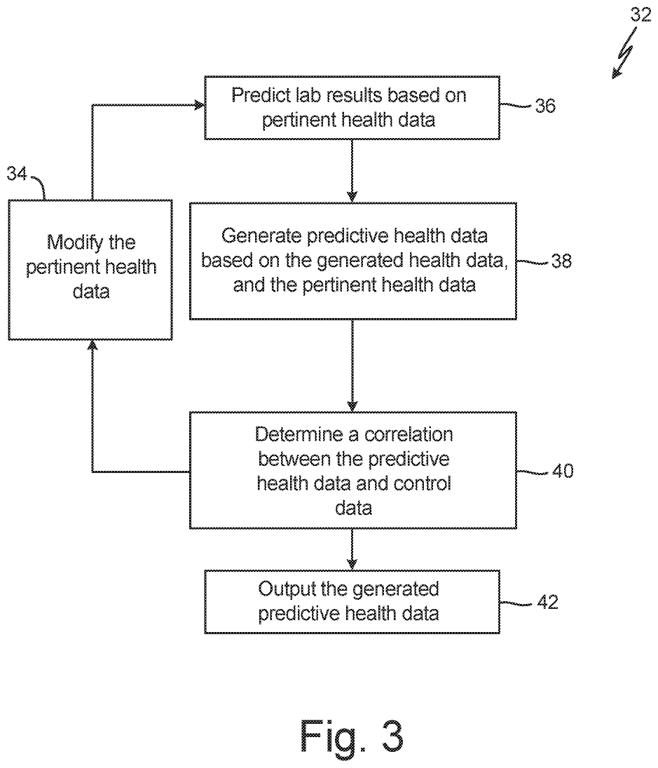

FIG. 3 is a flowchart illustrating method 32 of predicting future patient health. In some examples, method 32 can be utilized to generate treatment information as shown and discussed with regard to method 18, an example of which is illustrated in FIG. 2. Method 32 can generate treatment information for a patient that is based on the specific health parameters of that patient. As such, method 32 can provide health action recommendations that are identified, by the health evaluator 1 (FIG. 1), as corresponding with improved patient health.

Method 32 can be implemented by the health evaluator 1, among other computing options. Method 32 is a specific example regarding the iterative simulation of the health of a subject patient to arrive at desired treatment information for that patient. The health evaluator 1 is configured to analyze pertinent health data and generate predictive health data regarding a subject patient. The health evaluator is configured to implement one or more health prediction machine learning models to generate the predictive health data. It is understood that individual machine learning models can be trained to generate predictive health data regarding discrete aspects of patient health (e.g., generate predictive health data regarding a single lab result (e.g., creatinine), a single morbidity (e.g., hypertension), etc.). The multiple individual machine learning models can be considered to collectively form the health predictor model. Each machine learning model of the health predictor model can be trained as discussed above with regard to FIG. 1, among other options. In some examples, the health predictor models can be trained on baseline health data configured based on directed graphs to generate and output the predictive health data.

Method 32 is configured to generate information regarding the future health of a patient and provide treatment options that are configured to provide that desired future health for the patient. Method 32 is configured to generate predictive health data configured to provide enhanced (e.g., optimal, in some examples) health for a patient. Method 32 provides constrained optimization for determining patient health. The health predictor model is trained on baseline health data to determine patterns that correspond to the health response of persons similarly situated to the current patient. The generation of the predictive future health data can be constrained in that certain variables are modifiable to alter the pertinent health data while other variables are fixed and unmodifiable. In addition, the modifiable variables can be constrained to limit the modifications that can be made by the health evaluator. For example, the value of a modifiable variable can be limited in rate and/or value of the change from a base, starting value.

As discussed above, the health evaluator can generate predictive data regarding future patient health. The health evaluator is configured to identify various modifiable health parameters and introduce modifications to those modifiable parameters to generate predictive health data for a patient. The sets of predictive health data based on the modified health parameters identifies the health actions that can impact future patient health.

In step 34, pertinent health data for the patient is modified, such as by the health evaluator and/or the user altering one or more of the modifiable variables of the pertinent health data to generate modified pertinent health data. For example, lifestyle changes can be made by the patient (e.g., smoking cessation, increased exercise or other physical activity, maintaining or commencing a specified diet, etc.), the patient's lab results can be changed based on medication and/or lifestyle changes, follow ups with nurses and/or doctors can be scheduled or intervals between such checks can be changed, etc. In some examples, the health evaluator is configured to modify the health factors associated with the subject patient based on information generated by the health predictor model, such as information generated during training of the health predictor model. For example, the health predictor model can determine that certain health parameters are highly correlated incidences of a certain morbidity. The health evaluator can be configured to initially modify the highly correlated ones of the health factors to generate the modified health data, as such modification is likely to affect the subject morbidity.

The modified health data is utilized as the pertinent health data for the patient simulation conducted by the health evaluator. The factors forming the pertinent health data represent the state of the subject patient such that modifying the factors provides a modified patient profile that is analyzed by the health evaluator. The modified health data is analyzed to predict future health of the patient based on the modified patient profile. Treatment information can be generated based on the modified patient profile, such as by identifying the modified health parameters and forming health actions to address such modified health parameters.

In step 36, the health evaluator analyzes the modified health data to predict lab results for the patient. For example, the health evaluator can analyze current vital signs along with lab results, medical diagnoses, and medications (i.e., patient historical information, which can be provided as EMRs). The one or more health predictor models generate predictive health data regarding the modified patient and generate data regarding expected lab results for the patient based on the predictive health data. For example, the health evaluator can generate information regarding the expected level of the lab results at a period six months in the future, twelve months in the future, or at any other desired time period. The predicted lab results can be considered to be health data generated by the health evaluator, which can be referred to as generated health data.

In step 38, the health evaluator generates a set of predictive health data for the patient. The predicted lab results and the modified pertinent health data can be analyzed, such as by one or more health predictor models, to generate information regarding predicted diagnoses for the patient. For example, one or more machine learning models can be configured to generate the predicted diagnoses based on the predicted lab results and pertinent health data.

In some examples, the health evaluator can be configured to generate predicted diagnostic data as some or all of the predictive health data. The predicted diagnostic data is data regarding expected future diagnoses of the patient based on the generated health data and the pertinent health data. For example, the health evaluator can determine that the treatment actions for this patient may resolve two of three comorbidities while giving rise to an additional comorbidity. The health evaluator can generate a diagnosis count for the predicted diagnostic data, which is a count of the diagnoses indicated by the predicted diagnostic data. The predicted diagnostic data can include one or more expected morbidities of the patient given the pertinent health data and generated health data that is based on the pertinent health data.

In some examples, one or more health predictor models can be configured to generate information regarding predicted treatment actions for the patient. For example, the health predictor model can be configured to analyze unmodified pertinent health data of the patient with a desired result of the modified health data for that patient to generate health actions predicted to cause the subject patient to have the modified health data. For example, the modified health data can include removing a first prescription drug, modifying diet, and reducing BMI. The health evaluator can simulate treatments of the patient by various health actions embodied in the baseline health data to achieve an output correlating with the modified pertinent health data. Such simulated treatments can provide one or more treatment options to be output by the health predictor model.

The predictive health data is indicative of the future health of the patient given the base health data for that particular patient and based on the generated health data. The predictive health data can be built based on multiple sets of generated health data that is itself built based, at least in part, on other generated health data. In the example discussed, the predicted lab results can form a first set of generated health data, the predicted diagnoses can form a second set of generated health data based on the first set of generated health data, and the predicted treatment options can form a third set of generated health data based on the first and second sets of generated health data.

In step 40, the health evaluator determines whether the predictive health data corresponds with control data for the patient. The health evaluator can compare the predictive health data and the control data to determine whether the predictive health data provides target results for the patient. For example, the health evaluator can be configured to determine whether the predictive health data corresponds with target results based on the predictive health data minimizing the number of diagnoses, the predictive health data minimizing a composite score (e.g., based on an aggregated score in which various diagnoses are assigned values based on severity among other factors), decreasing (e.g., minimizing) patient costs, among other control data.

In some examples, method 32 can proceed to step 42 and output the predictive health data, such as by saving the predictive health data in the memory 2 of the health evaluator 1. In some examples, method 32 can be configured to proceed to step 42 in response to the health evaluator determining that the predictive health data corresponds with desired patient health at step 40. It is understood that the health evaluator 1 can be configured to output a first set of predictive health data as corresponding with the control data and method 32 can then proceed back to step 34 to iteratively modify the pertinent health data and generate further health information for the subject patient.

The health evaluator can be configured to iteratively generate predictive health data, as shown by the loop in steps 34-40, such as to identify one or more sets of health actions for improving patient health. The health evaluator can be configured to compile the multiple sets of predictive health data to form the treatment information. In examples where health evaluator generates additional sets of predictive health data, method 32 proceeds to step 34 to generate further modified pertinent health data. At step 34 the pertinent health is further modified to generate an additional set of modified pertinent health data. The additional modified health data is analyzed by the health predictor model to generate further sets of predictive health data regarding the patient. The health evaluator can be configured to generate multiple sets of predictive health data to provide multiple outputs having modifications to various ones of the modifiable health parameters, thereby providing an array of treatment options for improving patient health.

As such, the health evaluator can be configured to iteratively generate the predictive health data to generate a best set of predictive health data for a patient. For example, the health evaluator can generate a first predictive health data set based on the pertinent health data and generated health data. The health evaluator can execute one or more subsequent analyses to generate additional predictive health data sets based on one or more sets of modified pertinent health data. The health evaluator can be configured to compare the various sets of predictive health data to determine a best fit set for desired patient health from the various sets of predictive health data. The best fit set can be based on a correlation between the predictive health data and the control data. As discussed above, the health evaluator can determine the degree to which the predictive health data sets are correlated with desired patient health and/or can generate composite scores based on one or more parameters of the predictive health data and/or control data. In some examples, the health evaluator can generate a composite health score based on values assigned to various diagnoses or other control data (e.g., number or type of medications, number or type of lifestyle changes, etc.). In some examples, the health evaluator can be configured to generate predictive health data based on an iteration count, such that the health evaluator performs a set number of analyses and the best fit data is selected from that finite data set.

In some examples, the health evaluator is configured to determine the best fit based on the iteration count. For example, the health evaluator can maintain a count of the number of the predictive health data sets generated and the relative scores or values of those predictive health data sets as determined by the health evaluator. The health evaluator can compare the data set count to an iteration count threshold. For example, an iteration count threshold of fifty can indicate that the health evaluator stops generating additional, modified predictive health data after generating fifty sets of predictive health data. The health evaluator can be configured to reset the iteration count based on a predictive health data set being generated that better corresponds to desired patient health (e.g., indicating fewer diagnoses and/or a lower diagnosis score (e.g., based on scores assigned to various diagnoses); indicating lower expected costs; etc.) than a current best fit predictive health data set. The health evaluator can, in some examples, restart the count on a subsequent analysis. The health evaluator can identify a best fit one of the predictive health data sets based on the predictive health data set that best corresponds with desired patient health when the health evaluator reaches the iteration count threshold.

In step 42, the health evaluator outputs one or more predictive health data sets as treatment information. The predictive health data sets provide data that allows the patient and provider to make informed decisions on patient health. For example, the predictive health data generated by the health evaluator can indicate that a certain treatment course is likely to lead to the development of additional morbidities in certain patients, which development may not otherwise be apparent. The various predictive health data sets allow the patient and provider to select treatment actions that best correspond with that patient's desired health outcomes, rather than based on general reactions or responses within the population.

The health evaluator can output the modified health factors to the patient and/or provider along with the treatment information. The modified health factors identify the specific treatment goals for that patient that should lead to improved patient health. For example, a modified health factor can be a specific reduction in weight or BMI. The health actions generated for that patient will focus on achieving the modified health factors for that patient. In some examples, one or more treatment plans can be generated based on the information output by the health evaluator. For example, the modified health factors can indicate that frequent nurse visits will improve patient health, allowing such encounters to be scheduled which correlation may not be apparent on its face.

The health evaluator 1 and method 32 provide significant advantages. The health evaluator can predict lab results and generate an accurate medical diagnosis for a patient based on the current health of the patient and based on modifications in the health of the patient. The modifications can provide information regarding treatment actions, patient actions, etc. that will result in an optimal patient health outcome. By using the predictive model to predict how patient behavior, medications and interventions affect future vital signs and lab results, in order to predict future diagnoses, the most beneficial patient behavioral changes, medications and interventions can be identified that will also minimize the number of diagnoses of that patient. The predictive health evaluator model is configured to predict how patient behavior, medications, and interventions affect future vital signs and lab results, in order to predict future diagnoses, the most beneficial patient behavioral changes, medications, and interventions that provide an optimal treatment plan. The health evaluator analyzes all pertinent health data to generate predictive results, providing decision support to clinicians that may not be fully aware of other actions regarding the patient's health. Individual clinicians can analyze and modify suggested treatments based on the outputs from health evaluator thereby accounting for all aspects of the patient's health.

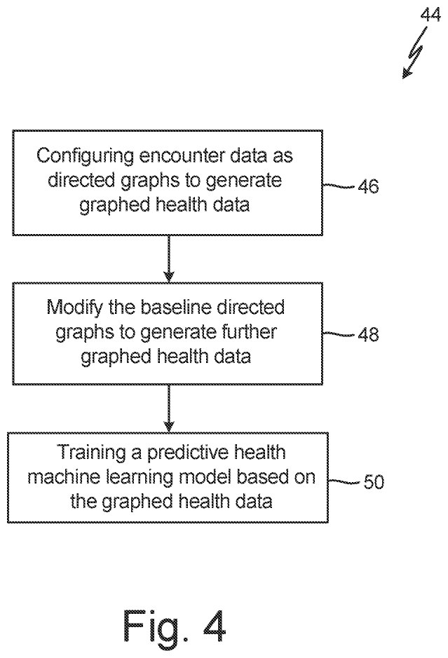

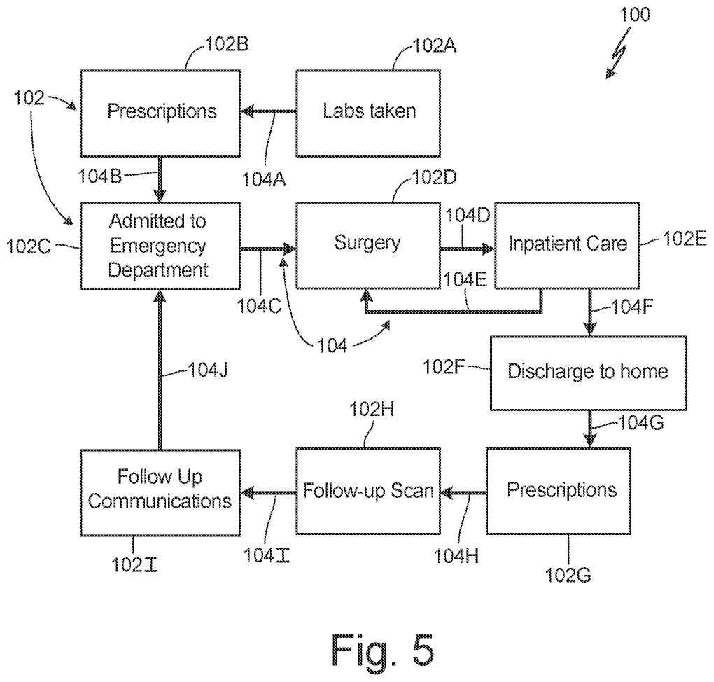

FIG. 4 is a flowchart illustrating method 44 of training a health prediction machine learning model. FIG. 5 is a diagram illustrating an example of a directed graph 100 for mapping patient-hospital encounters in accordance with the present disclosure. FIGS. 4 and 5 will be discussed together. Directed graph 100 is formed by nodes 102 and edges 104.

Encounters between a patient and a healthcare system can be defined graphically by directed graphs similar to directed graph 100. Directed graph 100 is representative of patient interactions within a healthcare system. In step 46, patient-healthcare system encounter data is configured as directed graphs 100. For example, EMRs can provide the encounter data that is configured as directed graphs 100. The EMRs can be considered to form some or all of the patient-healthcare system encounter data that is used to build the directed graphs. Health encounters can be defined in any desired manner, such that a single directed graph can map a single interaction between patient and provider or map a series of individual encounters over the course of days, weeks, months, years, etc. Any target healthcare encounter can be configured as directed graphs 100, in that one or more directed graphs can be built based on the entirety of treatments from a diagnoses to resolution of a condition (e.g., elevated blood pressure, elevated cholesterol, sprain, mental health disorders, etc.) or based on single-episode encounters (e.g., a single visit to the urgent care), among other options. In some examples, one or more individual healthcare encounters can be configured as a single directed graph 100. In other examples, a single healthcare encounter can be configured as multiple directed graphs 100. The one or more sets of one or more directed graphs built based on the encounter data can be considered to form graphed health data for training of the health prediction machine learning model.

As shown in FIG. 5, nodes 102A-102I (collectively herein “node 102” or “nodes 102”) and edges 104A-104J (collectively herein “edge 104” or “edges 104”) are components of directed graph 100. Nodes 102 and edges 104 provide information related to the interactions between the patient and the healthcare system. The information can be associated with the graph component as interaction data. Directed graph 100 is formed with edges 104 that are unidirectional. Some examples of directed graphs 100 do not include bidirectional edges 104 such that movement through directed graph 104 can occur in only a single direction along each edge 104.

Each node 102 is representative of one or more interactions between the patient and the healthcare system. For example, a node 102 can be indicative of a stay in the intensive care, a blood pressure reading, a blood test, a scan (e.g., magnetic resonance imaging (MRI), computerized tomography (CT), etc.), etc. Each node 102 can include interaction data regarding the actual events that took place and that are represented by that node 102. The interaction data includes information regarding the one or more events that occurred at that node 102. For example, each node 102 can include the interaction type (e.g., blood draw, scan, check in, medication prescribed, etc.), results of the interaction, the date that an interaction took place, the length of time of the interaction (e.g., a dwell time, such as the number of hours/days/etc. for a stay in the intensive care), specifics regarding the type of interaction (e.g., tonsillectomy, adenoidectomy, or a more general encounter descriptor such as “surgery”), the costs associated with an interaction, etc.

Edges 104 extend between and connect nodes 102. Edges 104 indicate the pathway of a patient through the healthcare system. In the example shown, edges 104 are unidirectional such that directed graph 100 has only a single direction of travel along edges 104. Edges 104 are pathways that connect sequentially adjacent nodes 102. Edges 104 can include interaction data associated with that edge 104. The interaction data can include temporal information and/or additional information regarding the period between nodes 102. For example, an edge 104 connecting a first node 102 indicating “medication prescribed” and a second node 102 indicating “medication received” can include the time delay between those two nodes 102. An edge 104 connecting a first node 102 indicating “CT scan ordered” and a second node 102 indicating “CT scan complete” can include the time delay between those two nodes 102. An edge 104 connecting a “surgery” node 102 and a “discharge to home” node 102 can indicate one or more other interactions occurring in the period between “surgery” and “discharge to home,” such as one or more stays in intensive care that occurred between those nodes 102 and various other interaction information, such as medications and doses given in the period between the connected nodes 102. In some examples, one or more edges 104 can include interaction data regarding patient activities between the nodes 102 (e.g., amount of daily exercise, changes in lifestyle factors (e.g., stopped smoking), etc.), among other types of interaction data.

While each edge 104 is itself unidirectional, directed graphs 100 can be configured such that edges 104 form loops between nodes 102 within directed graph 100. As shown in FIG. 5, a first node 102D representing a surgical procedure and a second node 102E representing a stay in the intensive care unit (ICU), illustrated in the example of FIG. 5 as “Inpatient Care”, are connected by multiple edges 104D, 104E to form a loop. A first one of the edges 104D extends from the first node 102D to the second node 102E, while a second one of the edges 104E extends back from the second node 102E to the first node 102D. Each of the edges 104D, 104E is unidirectional and together the multiple nodes 102D, 102E and edges 104D, 104E form a loop within the directed graph 100. The nodes 102 can include information regarding each encounter embodied at that node 102 (e.g., the “surgery” node can include interaction information regarding multiple surgeries occurring at different times). Configuring the directed graphs 100 as with unidirectional edges 104 that can be formed to include loops allows the health evaluator to ascertain patterns and identify relationships among the encounter data. The unidirectional, and in some examples looping, configuration of the edges 104 forming directed graphs 100 generates patterns that facilitates training of the health predictor machine learning model to identify patterns from the EMRs that are predictive of future patient health.

The directed graphs 100 are built based on the EMRs, such as by the health evaluator 10 (FIG. 1) executing the health graphing module 14 (FIG. 1). EMRs provide hundreds or thousands of records from a rich, real-world data environment. Configuring the baseline health data as directed graphs 100 provides quantifiable data for training the health predictor models. Various health data regarding the patient is known at precise locations along the graph, such as the number of diseases, whether a condition (e.g., hypertension, diabetes, congestive heart failure, etc.) is controlled, etc. Patient health can be quantified based on known patient events (e.g., diagnoses, medications, whether a condition is controlled, etc.) that are located at known points on the directed graph 100. The health predictor models can be configured to determine patterns in the baseline data and generate scores for variables based on the baseline data.

Directed graph 100 includes measures of patient health embedded as data in various components of the directed graph 100. The health of a patient can be quantified at various points that can be represented in the directed graph 100. For example, the health evaluator can determine relative severities of diagnoses, relative severities of conditions, relative importance of lab results, relative severities of drug interactions, etc. based on the baseline health data. In some examples, encounter data can be quantified and provided to the health evaluator, such as by the user quantifying the data.

In some examples, the health evaluator can learn relative interactions, severities, etc. based on the directed graphs and can additionally or alternatively generate and assign scoring metrics to various health conditions, treatments, patient lifestyle factors, etc. In some examples, baseline scoring metrics can be assigned to components of the directed graphs and/or the interaction data prior to providing the health data to the health evaluator as a set of baseline training data. In additional or alternative examples, the health evaluator can be configured to generate scoring metrics and/or modify the baseline scoring metrics. For example, the health evaluator can be configured to generate ranking data for various of the health data. In some examples, the health evaluator can generate scores or other indicators of rank for various morbidities with the scores based on the severity of each morbidity. For example, the health evaluator can identify patterns that indicate that certain morbidities inversely correspond with patient lifespan and can quantify the morbidities based on relative severity.

The health evaluator can determine relationships between factors of the health data. For example, the health evaluator can determine the relative impact on health for a first combination of a first morbidity and a second morbidity relative to a second combination of the first morbidity, a third morbidity, and a fourth morbidity. The health evaluator can determine that the second combination, having three comorbidities, is actually better for overall patient health as opposed to the first combination, having two comorbidities, which relationship would not be apparent from a base comparison of comorbidity count. The health evaluator can determine that a treatment may resolve some morbidities and give rise to other morbidities, among other data predictive of future patient health.

As shown in FIG. 5, an example sequence of events associated with a patient-healthcare system encounter may include a lab test being performed, medication being prescribed, admission to an emergency department, performance of one or more surgeries, moving of the patient to inpatient care, performance of further surgery and subsequent moving of the patient to inpatient care (shown as a loop in FIG. 5), discharge of the patient to home, medication being prescribed to the patient, a follow-up scan, follow-up communications with the provider, and possibly an additional admission to the emergency department. It is understood that the sequence shown in FIG. 5 is shown at one resolution relative to other directed graphs built based on EMRs. The directed graphs 100 built based on the EMRs and used to build the baseline health data can include hundreds or thousands of nodes 102 and edges 104.

The sets of directed graphs built based on the patient-healthcare system encounter data can be considered to form graphed health data. In step 48 (FIG. 4), the graphed health data is modified to generate additional training data for the health prediction model, in that the baseline directed graphs are modified to generate additional directed graphs 100. The graphed health data can be manipulated to introduce alterations to the directed graph 100 to thereby form one or more sets of modified directed graphs. The modified directed graphs form manipulated health data that can form a portion of the graphed health data for training of the health prediction machine learning model.

The modified directed graphs can include variations to the directed graphs themselves and/or to the interaction data. For example, perturbations to time and/or resolution can be introduced to the directed graphs 100 forming the graphed health data. One or more additional sets of directed graphs can be generated as the modified graph data. The nodes 102 and/or edges 104 and/or the interaction data of a directed graph 100 can be manipulated to generate one or more sets of additional directed graphs 100. The perturbations alter the base graphed health data to facilitate the matching of similar sequences across patient populations.

In some examples, resolution perturbations can be introduced to generate the modified directed graphs. The modified directed graphs can be generated at a higher resolution with more detail, and/or at a lower resolution with less detail. For example, more or less details of an incident can be included in the interaction data embedded in a graph component, more or less nodes 102 and/or edges 104 can be included in the modified directed graphs, etc.

The encounter data can be manipulated to alter the resolution of the directed graph 100. The graph components are manipulable to increase or decrease the resolution of the directed graph 100. For example, interactions can be consolidated within nodes 102 and/or edges 104 to provide lower resolution directed graphs or spread between multiple nodes 102 and/or edges 104 to provide higher resolution directed graphs. In some examples, interaction data for multiple interactions (e.g., multiple surgical interactions) can be correlated based on a common base interaction, such as the event type being a “surgery.” The interaction data for each event can be consolidated in a common node 102 in lower resolution directed graphs 100. The interaction data for each event can be dispersed across separate nodes 102 in higher resolution directed graphs 100. For example, multiple graph components can be consolidated to a single consolidated graph component in lower resolution directed graphs, while a single graph component can be separated into one or more separate graph components in higher resolution directed graphs.

When the interaction data is combined in lower resolution directed graphs 100, the interaction data can be maintained separately within portions of the memory allocated to the graph component itself (e.g., as a separate data subsection or subdivision). For example, the “Surgery” node 102 can include first interaction data related to a first surgery and second interaction data related to a second surgery. The first interaction data and the second interaction data are generated based on discrete interactions (e.g., the individual surgeries), which discrete interactions can be viewed as a single interaction (e.g., as a single node 102) in a lower resolution directed graph 100 and can be viewed as separate interactions (e.g., as multiple nodes 102) in a higher resolution directed graph 100. The interaction data can be combined or separated to form directed graphs having differing resolutions.

In the example shown in FIG. 5, the node 102E indicating “Inpatient Care” may actually represent a series of interactions all relating to a general inpatient care interaction, such as a stay in intensive care, a subsequent stay in a recovery room, a surgery, transfer back to intensive care, and another stay in a recovery room. A directed graph having a higher resolution than directed graph 100 shown in FIG. 5 can be built by expanding the “Inpatient Care” node 102E to multiple nodes, such as one or more nodes 102 for each of the intensive care stays, the surgery, and/or the recovery room stays, connected by multiple additional edges 104. The node 102I indicating “Follow-Up Communications” can be represented at a higher resolution by indicating the type of communication (e.g., email, phone call, text, etc.), a count (e.g., number of communications), content (e.g., data from patient, additional or alternative treatment options provided, etc.), the communicator identity (e.g., doctor, nurse, physical assistant, administrative, etc.). A directed graph having lower resolution than directed graph 100 shown in FIG. 5 can be generated by combining various of the nodes 102. For example, the “Admitted to Emergency Department,” “Surgery,” and “Inpatient Care” nodes 102C, 102D, 102E can be consolidated to a single “Inpatient Treatment” node 102, in one example of a lower resolution directed graph 100.

Building directed graphs 100 at multiple resolutions enables training of the health evaluator to identify (e.g., sometimes referred to as “learning” in the context of machine learning or other predictive algorithms) both gross and fine patterns that may be predictive of patient health. For example, the lower resolution graphs reveal patterns that may be common across cohorts of patients, which may be less apparent in high resolution graphs. The higher resolution graphs reveal fine patterns and nuanced relationships that may not be apparent in the lower resolution graphs.

The directedness of directed graphs 100 is based on the temporal order of the events. Perturbations in time alter encounter sequences and/or temporal distance to facilitate the matching of similar sequences across patient populations. Edges 104 extend between sequential nodes 102. The sequences of the interactions or events can be varied to generate additional or alternative modified directed graphs. Time perturbations can be introduced to the directed graph 100 to generate modified directed graphs. The time perturbations can vary the time data and/or ordering of the directed graph 100. Time perturbations can be introduced to account for variances in the actual ordering of events based on the EMRs. In some examples, time perturbations can be applied to reorder the nodes 102. For example, the actual ordering of the interactions for a first patient may vary from the actual ordering of the interactions for a second patient. Reordering the interactions, such as by reordering the sequence of the nodes 102, generates manipulated health data that increases accuracy and reliability of the resulting output from the health evaluator. In some examples, the timeline of an encounter and/or within interactions in an encounter can be subjected to random perturbations to generate the additional modified directed graphs 100 and build the modified health data.

In some examples, the time perturbations can vary or otherwise alter the time periods between interactions or events to generate the modified directed graphs. The time perturbations can be applied such that events that are sequentially similar, though with differing dwell times or periods, are highly correlated by the health evaluator. As such, the directed graphs can be modified by introducing perturbations to the resolution of the data. For example, a follow-up scan may take place for a first patient six months after discharge from the hospital while the same or similar follow-up scan may take place for a second patient two months after discharge from the hospital. The interaction data regarding the time delay between the discharge event and the follow up scan event can be manipulated to indicate a range (e.g., 1-8 months, etc.), an upper bound (e.g., less than 12 months), or lower bound (e.g., more than 2 weeks), etc. thereby providing a broader, more robust data set for training the health predictor machine learning models.

The time data portion of the graphed health data can be manipulated to generate one or more sets of modified directed graphs. Interactions between the patient and healthcare system can be analyzed and condensed based on the nature of the interactions themselves. For example, a dispersed interaction is an interaction that has multiple discrete events that all relate to a single temporal interaction point but that occur over a span of hours, days, weeks, months, etc. Such a dispersed interaction relates to the single temporal interaction point, but the discrete events that make up that dispersed interaction could be graphed as discrete events occurring at separate points in time. One option is to graph the discrete events as individual nodes 102 connected by edges 104, though such dispersed data can be misleading regarding the nature of the encounter and the condition of the patient. In some cases, additional interactions may occur in the time period between the discrete events forming the dispersed interaction, further obscuring the sequential nature of the graphed encounter.

The data regarding dispersed interactions can be consolidated as interaction data related to the single event. For example, that single interaction can be consolidated to and represented by a graph component of the directed graph 100 (e.g., as a node 102, an edge 104, or combination thereof). The various discrete events can be associated with a true date, which is the single temporal interaction point to which the discrete events actually relate. The time data for each of the discrete events can be modified such that each of the discrete events is correlated with the true date. For example, assume a patient's blood is drawn on day one, results are obtained on day fifteen, and a diagnosis is made on day thirty-five. While the single interaction may appear to be three discrete events occurring at three different points in time, each of the three discrete events actually relate to the patient condition and health on day one, which is when blood samples were taken. In such an example, day one can be considered to be the true date for the single interaction formed by the discrete events. A graph component, such as a node 102, is generated based on the combined interaction data and the interaction data for that graph component is associated with the true date. Manipulating the graph data to generate such directed graphs with discrete events associated with the single temporal interaction point improves the accuracy and reliability of the health evaluator. Associating health data with the actual point in time (i.e., the true date) provides accurate data to facilitate the health predictor machine learning model accurately and efficiently learning various patterns, increasing the accuracy of the output from the health evaluator.

In step 50, the directed graphs generated based on the encounter data and the directed graphs generated based on the manipulated encounter data are utilized to train the health prediction machine learning model to generate predictive outcomes regarding patient health, such as by the health evaluator 10 executing the machine learning training module 10. Sets of features are generated based on the graphed health data to form the baseline health data for training the health predictor models based on the directed graphs. The health predictor model can be trained similar to the discussion above with regard to FIG. 1. The data utilized to train the health prediction model is generated, at least in part, based on the graphed health data, such as by the health evaluator 10 executing the health graphing module 14. The baseline health data utilized to train the health predictor model is generated based on the graphed health data. The health predictor model utilizes machine learning to analyze, understand, and/or respond to patient data. The application of machine learning algorithms to input in the form of directed graphs 100 can enable EMRs to be converted into data that can be processed and evaluated for patterns. By analyzing a selection of features generated based on directed graphs 100, machine learning models can be trained to recognize, classify, and react to pertinent health data.

The baseline health data is generated based on at least in part on the EMRs of the patients forming the patient population. The baseline health data for training the health predictor model can be based on sets of record features that are extracted, directly or indirectly, from the sets of EMRs of each patient in a patient population associated with a subject health parameter. Properties of the directed graphs are quantified to form graphed health data that forms additional features of the baseline health data for training the health predictor model. The quantified properties can include information regarding the component of the graphs themselves, such as the number of nodes, the number of edges, the ratio of nodes to edges, the lengths of edges, the encounter count at each node, among other options regarding the directed graph and information contained in the directed graph. The baseline health data can be formed by sets of features that include both the record features taken directly from or derived from the sets of EMRs and the graph features that are the quantified parameters of the directed graphs. It is understood that in some examples both the record features and the graph features can be considered to be derived from the EMRs as the directed graphs can be built based on encounter data from the EMRs.

The baseline health data is built based at least in part on the sets of features generated based on the graphed health data. As such, the baseline health data can be considered to be formed at least partially by the graphed health data. In some examples, the features extracted from the directed graphs can form additional data columns in the feature table for the baseline health data, supplementing the sets of record features. In some examples, a first set of baseline health data is generated based on features extracted from a first set of graphed health data. A second set of baseline health data can be generated based on modified graphed health data for which perturbations in time and/or resolution are introduced to modify the first set of graphed health data. The health predictor models can be trained in a first stage based on the sets of features of the first baseline health data and trained in a second stage based on the sets of features of the second baseline health data. In such an example, the first and second baseline health data vary in the features extracted from the graphed health data for each set of baseline health data. In some examples, the health predictor model is trained on baseline health data generated based on the directed graphs and based on modified directed graphs, such as those that have had time and/or resolution perturbations introduced. The sets of features forming the baseline health data can include graph features from the directed graphs and modified graph features from the modified directed graphs. The baseline health data can include features from as many or as few sets of directed graphs as desired.

In some examples, the health predictor model can be trained in stages based on the graphed health data. For example, a first set of graphed health data can be generated based on a first set of directed graphs. A first set of baseline health data is generated based on the first set of graphed health data and the health predictor model is trained on the first set of baseline health data. A second set of graphed health data can be generated based on modifications made to the directed graphs forming the first set of directed graphs. As such, the second set of graphed health data generated based on a second set of directed graphs different from, but based on the first set of directed graphs. A second set of baseline health data is generated based on the second set of graphed health data and the health predictor model is retrained on the second set of baseline health data.

Training or retraining of the machine learning model to account for aspects such as time and resolution perturbations (e.g., based on the manipulated graphed health data) can increase accuracy and reliability of the resulting output from the model. In some examples, the health evaluator can determine that certain sequences and/or portions of patient-healthcare system encounters are important relative to certain patient health factors and unimportant relative to those patient health factors.

Diagnostic data (e.g., a diagnosis of diabetes, hypertension, congestive heart failure, etc.; whether the diagnosed disease is controlled or not; etc.) forms a portion of the interaction data. As such, the diagnostic data for a patient is known at various points within the directed graph 100 of a patient encounter. The status of a patient (e.g., count/type of comorbidities, medications, lifestyle factors such as smoking and exercise, etc.) is known at various points in time and that patient data can be recorded as interaction data at one or more nodes 102 and/or edges 104 of the directed graph 100. The diagnostic data facilitates training of the health predictor models based on real world outcomes, facilitating generating data regarding future patient health. The health evaluator is configured to predictively determine future patient health. The health predictor models of the health evaluator are trained based on the baseline health data. The health prediction model can be trained utilizing supervised training methods, among other options. Building directed graphs 100 and then extracting features from such directed graphs to generate the baseline health data facilitates the machine learning software solution learning like-sequences of patient-hospital interactions and behaviors that are predictive of patient health in a computationally efficient way.

Building the baseline health data as multiple data sets based on features of directed graphs and modified features that introduce perturbations to form manipulated graphed health data facilitates the use of data that is collected at high and low frequencies and data with many interaction points and having random orders, which is typically of EMRs. Also, varying the graph resolution and making perturbations in the graphs and then quantifying the properties of these modified graphs enables the development of more robust predictive models that are less likely to overfit the original data.

The health evaluator can be configured to generate treatment information, such as by executing the patient simulation module 12 (FIG. 1). In such examples, patient data (e.g., age, race, economic status, lifestyle factors such as smoking and drinking, comorbidities, EMRs specific to the patient etc.) and treatment data (e.g., one or more of lifestyle changes, additional or different medications, one or more procedures, frequency of follow ups, etc.) can form pertinent health data of the patient. The treatment data can be based on the treatment plan determined by the provider to achieve the treatment goal (e.g., minimizing the number of comorbidities, lowest cost, fewest medications, etc.). For example, a patient may have a diagnosis of hypertension along with three other comorbidities and several current prescriptions, and the treatment goal may be having the patient's blood pressure controlled within the next twelve months.

The health predictor models are trained to generate predictive health data for the patient based on the patient data and the proposed treatment data for the patient. The patient data and proposed treatment data are provided to the health evaluator and the health evaluator generates predictive health data for the patient. The health evaluator can be configured to generate predicted treatment outcomes based on the patient data and treatment data. In some examples, the health evaluator can indicate whether the treatment goal will be achieved along with other predictive health data. In additional or alternative examples, the other predictive health data can include whether comorbidities are expected to increase or decrease, inflection points in treatment plan (e.g., when various comorbidities are expected to be clinically controlled or arise), etc.

Method 44 and the health predictor models trained based on method 44 provide significant advantages. The health evaluator generates predictive health data based on real world EMRs that are utilized to train the health evaluator. The health evaluator is trained on real world EMRs that provide a rich data set that can include hundreds or thousands of interactions that may not be captured in the clinical trials. Moreover, the breadth and depth of the health data in the EMRs facilitates building a robust health evaluator that can accurately generate predictive health data for patients.

Training of the health predictor models on baseline data configured as directed graphs 100 provides significant advantages. It is understood that, while the baseline health data is described as including modified health data, in some examples method 44 can proceed directly from step 46 to step 50 such that the health predictor models are trained based on sets of features generated based on the directed graphs 100 built directly based on the patient-healthcare system encounter data. The baseline health data for training the health predictor models is generated based on patient-hospital encounters that are configured as directed graphs 100. Directed graphs 100 (unlike hash tables) are visually intuitive and easily interpreted, making them ideal for use in clinical decision support. Also, the methods described herein do not require specialized computational resources beyond what are already commonly used in predictive healthcare models (e.g., computers with appropriately configured hardware/processors). The software solution is platform independent and addresses a problem specifically arising in the realm of EMRs and predictive patient health. Configuring EMRs as directed graphs facilitates training of the health predictor models to determine aspects of future patient health.

Properties of the directed graphs 100 can be quantified for analysis by the health predictor machine learning model for the development of predictive analytics. The directed graphs 100 that represent patient-hospital encounters are built and analyzed at multiple resolutions, so that both gross and fine patterns that may be predictive of patient health can be learned. Also, the directed graphs 100 can be built with time perturbations that rearrange the sequence of encounters to facilitate the matching of sequences across patient populations. As a result, useful and intuitive predictive analytics is obtained to support clinical decision-making, without requiring special resources or equipment. Configuring EMRs as directed graphs 100 generates an interpretable form of EMRs that can be analyzed to simulate and predict patient health.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the present disclosure.

1. A method of predicting future patient health, the method comprising:
receiving, by a machine learning model trained to identify a future parameter status of a subject health parameter based on baseline health data, pertinent health data regarding a patient, wherein the machine learning model is implemented on a health evaluator having memory and control circuitry, and wherein the baseline health data is generated based on sets of features extracted from sets of electronic medical records of each patient of a patient population associated with the subject health parameter;
analyzing the pertinent health data, the machine learning model, to generate predictive health data regarding an expected patient condition;
generating, by the health evaluator, treatment information for the patient based on one or more sets of the predictive health data; and
outputting, by the health evaluator, the treatment information.

2. The method of claim 1, further comprising:
modifying, by the health evaluator, the pertinent health data to generate modified health data; and
analyzing the modified health data, by the machine learning model, to generate the predictive health data regarding the expected patient condition.

3. The method of claim 2, further comprising:
generating the pertinent health data based on patient data and treatment data.

4. The method of claim 2, further comprising:
identifying first factors of the pertinent health data as modifiable; and
modifying one or more of the first factors to generate the modified health data.

5. The method of claim 2, wherein:
generating, by the health evaluator, the treatment information for the patient based on the one or more sets of the predictive health data includes:
identifying, by the health evaluator, a best fit set of the predictive health data based on a count of predicted diagnoses for each set of the predictive health data; and
outputting, by the computing device, the treatment information includes:
outputting, by the computing device, the best fit set of the predictive health data as the treatment information.

6. The method of claim 1, wherein analyzing the pertinent health data, by the machine learning model, to generate the predictive health data regarding the expected patient condition includes:
comparing, by the health evaluator, the generated predictive health data and control data regarding the patient; and
determining, by the health evaluator, a correlation between the generated predictive health data and target patient health based on the comparison.

7. The method of claim 6, further comprising:
modifying the pertinent health data to generate a plurality of sets of modified health data;
generating a plurality of additional sets of predictive health data based on the plurality of sets of modified health data;
comparing, by the health evaluator, each set of the plurality of additional sets of predictive health data and the control data;
determining, by the health evaluator, correlations between each set of the plurality of additional sets of predictive health data and the target patient health based on the comparison between each set of the plurality of additional sets of predictive health data and the control data.

8. The method of claim 7, further comprising:
applying, by the health evaluator, a scoring metric to each set of predictive health data to generate scored sets of predictive health data.

9. The method of claim 8, wherein outputting, by the computing device, the treatment information includes identifying, by the health evaluator, a best fit one of the scored sets of predictive health data based on the scoring metric.

10. The method of claim 9, wherein identifying, by the health evaluator, the best fit one of the scored sets of predictive health data based on the scoring metric includes:
generating, by the health evaluator, a composite score for each set of predictive health data forming the scored sets of predictive health data; and
identifying, by the health evaluator, the best fit one of the scored sets of predictive health data based on the composite scores.

11. The method of claim 8, further comprising:
generating, by the health evaluator, a composite score for each set of predictive health data forming the scored sets of predictive health data; and
ranking, by the health evaluator, the scored sets of predictive health data based on the composite scores for each set of predictive health data forming the scored sets of predictive health data.

12. The method of claim 1, wherein analyzing the pertinent health data, by the machine learning model, to generate the predictive health data regarding the expected patient condition, comprises:
generating, by the machine learning model, predicted laboratory results for the patient based on the pertinent health data; and
generating, by the machine learning model, predicted diagnoses based on the predicted laboratory results and the pertinent health data.

13. The method of claim 12, further comprising:
generating, by the machine learning model, predicted health actions based on the predicted diagnoses, the predicted laboratory results, and the pertinent health data; and
building the treatment information based on the predicted health actions.

14. The method of claim 2, wherein analyzing the pertinent health data, by the machine learning model, to generate the predictive health data regarding the expected patient condition, comprises:
generating, by the machine learning model, predicted laboratory results based on the modified health data; and
generating, by the machine learning model, predicted diagnostic data as at least a portion of the predicted health data, wherein the predicted diagnostic data includes one or more expected morbidities of the patient based on the predicted laboratory results and the pertinent health data.

15. The method of claim 14, further comprising:
comparing, by the health evaluator, the predicted diagnostic data and control data to determine whether the predictive health data corresponds with target patient health, wherein the control data is pre-generated and provided to the health evaluator, and wherein the control data is based on treatment goals for the patient.

16. The method of claim 15, wherein comparing, by the health evaluator, the predicted diagnostic data and the control data to determine whether the predictive health data corresponds with the target patient health comprises:
comparing, by the health evaluator, a diagnosis count of the predicted diagnostic data with a target diagnosis count of the control data; and
determining, by the health evaluator, that the predictive health data corresponds with the target patient health based on the diagnosis count of the predicted diagnostic data being one of equal to and less than the target diagnosis count of the control data.

17. A method of generating future health information, the method comprising:
(i) receiving pertinent health data, including vital signs and electronic medical records, associated with a subject patient;
(ii) setting initial design variables representing modifiable risk factors associated with the subject patient;
(iii) predicting laboratory results associated with the patient, by a machine learning model trained on baseline health data that is generated based on sets of electronic medical records of patients in a patient population, based on the received pertinent health data and the initial design variables;
(iv) generating predictive health data for the subject patient, by the machine learning model, based on the predicted laboratory results and the pertinent health data;
(v) comparing, by a health evaluator having control circuitry and memory and configured to implement the machine learning model, the predictive health data and control data to determine whether the predictive health data corresponds with desired patient health; and
(vi) outputting the predictive health data as treatment information based on the health evaluator determining that the predictive health data corresponds with desired patient health.

18. The method of claim 17, wherein generating predictive health data for the patient, by the machine learning predictive model, based on the predicted laboratory results and the pertinent health data, comprises:
generating predicted diagnostic data for the patient, by the machine learning model, based on the predicted laboratory results.

19. The method of claim 17, further comprising:
introducing modifications to the initial design variables to generate modified design variables; and
iteratively repeating steps (iii)-(vi) based on the modified design variables to generate a plurality of sets of predictive health data.

20. The method of claim 19, wherein iteratively repeating steps (iii)-(vi) based on the modified design variables to generate the plurality of sets of predictive health data includes:
iteratively repeating steps (iii)-(vi) based on an iteration threshold; and
stopping continued simulation based on the modified design variables based on an iteration count reaching or exceeding the iteration threshold.
